(19) United States
(12) Patent Application Publication
(10) Pub. No.: US 2004/0019265 A1
(43) Pub. Date: Jan. 29, 2004

(54) Fiducial marker devices, tools, and methods

(76) Inventors: Rudy A. Mazzocchi, Indian Harbor Beach, FL (US); Matthew S. Solar, Indialantic, FL (US); David Lee, Melbourne Beach, FL (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/206,884

(22) Filed: Jul. 29, 2002

(51) Int. Cl. A61B 5/05

(52) U.S. Cl. 600/407

(57) ABSTRACT

This document discusses, among other things, a fiducial marker assembly that includes an internally engagable base. The base is sized and shaped to be mounted flush to or recessed from an outer surface of a patient's skull, thereby reducing or avoiding patient discomfort. The fiducial marker assembly includes an imagable locator and a registration receptacle. A base insertion instrument is engaged into the interior of the base to attach the base to the patient's skull. A guide tube and scalp-stabilizer are provided for assisting in the base-attachment procedure. A trajectory guide can be coupled to the base. An alternative conformal cap to a raised fiducial marker base is also discussed.

### FIELD OF THE INVENTION

This document relates generally to imaging a patient for performing surgical intervention, and more specifically, but not by way of limitation, to fiducial marker devices and associated tools and methods.

### BACKGROUND

Fiducial markers that can be located and recognized by an imaging system are useful in neurosurgery and other applications. For example, in one technique, multiple fiducial markers are screwed into the patient's skull to define recognizable landmarks that appear on a preoperative image of the patient's brain. Such a bone-anchored fiducial marker typically includes an externally threaded bone-screw portion, which is driven into the skull, and a threaded shaft that rises up and out of the skull from the bone-screw. The threaded shaft typically receives a screwed-on imagable sphere that is visible on a magnetic resonance imaging (MRI) image or computed tomography (CT) image. The multiple fiducial markers on the patient's skull define landmarks on preoperative images that are useful to the physician for planning entry coordinates and a trajectory to a target location in the brain. An image-guided workstation uses these preoperative images and planning to guide the neurosurgeon while actually performing the subsequent surgical procedure.

After the preoperative planning phase, the patient is brought into the operating room so that the planned surgical procedure can be performed. On the operating table, the patient's skull is clamped in a head-frame or otherwise immobilized. In order to use the preoperative images provided by the image-guided workstation to guide the surgeon during the procedure, the patient's skull must first be “registered” to the preoperative images. The registration creates an association between (1) the actual physical location of the fiducial markers on the patient's skull in the operating room and (2) the locations of the images of the fiducial markers visible on the preoperatively-obtained images.

According to one registration technique, a “wand” is used to perform the registration. The wand includes multiple light-emitting diode (LED) locators or reflective locators, which are visible to an infrared or other camera in the operating room. The camera is connected to the image-guided workstation. The locators define the position of the wand in the operating room, including the position of a sharp tip portion of the wand, which is in a known physical relationship to the locators. To register the patient, the imagable spheres are unscrewed from the fiducial marker shafts, and replaced by respective “divots” that are sized and shaped to receive the wand tip. These divots are screwed onto the fiducial marker shafts, such that the maximum depression point of the tip corresponds to the same location as the center of the imagable sphere when the imagable sphere was screwed onto the fiducial marker shaft. A reference divot is also present in the operating room at a known location, such as on the operating table or head-frame. During the patient registration process, the surgeon touches the wand tip to the reference divot, and then to each fiducial marker divot. This permits the image-guided workstation to correlate the actual physical location of the patient's skull to the preoperative images. The physician can then use the wand, in conjunction with the image-guided workstation, to locate an appropriate entry point and trajectory to the target in the brain.

One problem with the above registration procedure is the discomfort caused to the patient by the presence of the fiducial marker shaft extending upward from the bone-screw portion of the fiducial marker for receiving the screw-on imaging sphere and the screw-on divot. The upwardly-extending fiducial marker shaft can cause irritation to the patient's scalp. The presence of external threads on the shaft may increase the level of this irritation. Moreover, because there may be a long time period between preoperative imaging and the subsequent surgical procedure, the patient's scalp may be sewn up during the interim. Thus, the patient may experience such discomfort for an extended period of time. For these and other reasons, which will become apparent upon reading the following detailed description and viewing the drawings that form a part thereof, the present inventors have recognized an unmet need for fiducial marker devices, tools, and methods that reduce or avoid patient discomfort.

### SUMMARY

This document discusses, among other things, fiducial marker devices and associated tools and methods. In a first example, the document discusses a system. In this example, the system includes an anchoring base. The base is sized and shaped to be implanted within in a patient's skull such that a top portion of the base is flush to or recessed from an outer surface of the patient's skull. The base includes an externally threaded outer portion extending from the top portion of the base. The base also includes an engagable base receptacle extending from the top portion of the base. The system also includes a locatable fiducial marker. The locatable fiducial marker includes a shaft, a portion of which is sized and shaped to be received and engaged into the base receptacle. The system also includes a registration receptacle. The registration receptacle includes a shaft, a portion of which is sized and shaped to be received and engaged into the base receptacle.

Variations on this example include, but are not limited to, a base in which the top portion of the base includes at least one tool-receiving receptacle in addition to the base receptacle. Another variation includes a top portion of the base that includes at least one slot. A further variation includes a plurality of screwdriver slots. Examples of the fiducial marker include, without limitation, such a marker that is imagable by at least one of: magnetic resonance imaging (MRI), computed tomography (CT), X-ray radiography, a light detector, and an electromagnetic field detector. In one variation, the system includes a plug sized and shaped to be received in base receptacle. In a further example, the plug is constructed to allow the plug to be press-fit into the base receptacle. In yet a further example, the plug is sufficiently compliant to allow the plug to be removed from the base receptacle by inserting a tool into the plug to assist in pulling the plug out of the base receptacle. In yet another example, the plug is sized and shaped to provide a top surface that is substantially flush with the top surface of the base receptacle.

In another variation, the system further includes a base insertion tool. The base insertion tool includes an engaging portion sized and shaped to be received and engaged within the base receptacle. The base insertion tool also includes a shaft, which is coupled to and extending outwardly from the engaging portion.

In another variation, the system further includes a guide tube. The guide tube includes a lumen extending longitudinally therethrough. The lumen is sized and shaped to allow the base to pass through the lumen. The guide tube includes a beveled distal tip, which is sized and shaped to align the guide tube lumen to a portal in the patient's scalp. In a further variation, the guide tube includes a (fixed or slidable) flange extending outwardly from the guide tube near the beveled distal tip, at a (fixed or slidable) distance from the beveled distal tip that is selected such that the flange stabilizes a portion of the patient's scalp near the portal in the patient's scalp when the beveled distal tip of the guide tube is pressed into a portion of the portal in the patient's scalp. Thus, in one example, the flange is longitudinally slidable along the guide tube to select the distance from the beveled distal tip to stabilize a portion of the patient's scalp.

In another variation, the system further includes a trocar sized and shaped to be received within the lumen of the guide tube. In yet another variation, the system further includes a drill bit. The drill bit is sized and shaped to be received within the lumen of the guide tube. The drill bit includes an outer diameter that is sized and shaped to create a portal in the patient's skull. The portal is sized to receive the externally threaded portion of the base in a self-tapping manner. In one example, the base receptacle is internally threaded to provide an engagement mechanism for the base. In another example, the base receptacle includes a male or female snap-fit coupling to provide an engagement mechanism for the base.

In a second example, this document discusses, among other things, a method. The method includes threading or otherwise inserting an anchoring base into a patient's skull such that a top portion of the base is flush with or recessed from an outer portion of the patient's skull. A locatable fiducial marker is coupled to a first receptacle in the base. An image of the patient's skull is obtained, such that the locatable fiducial marker is apparent on the image. The locatable fiducial marker is removed from the first receptacle in the base. A registration receptacle is coupled to the first receptacle in the base. A portion of the registration receptacle is in a predetermined spatial relationship to a portion of the locatable fiducial marker when the locatable fiducial marker was coupled to the base. A location of the portion of the registration receptacle is registered to the portion of the locatable fiducial marker that is apparent on the image.

Among the other variations, threading the anchoring base into a patient's skull may comprise: engaging an internal portion of the first receptacle in the base onto a tool; screwing external threads of the base into the patient's skull; and, disengaging the tool from the first receptacle in the base. In another variation, the engaging the internal portion of the first receptacle in the base onto a tool includes threading internal threads of the first receptacle in the base onto external threads on the tool. In another variation, the engaging the internal portion of the first receptacle in the base onto a tool includes snap fitting an internal portion of the first receptacle in the base onto an external portion of the tool.

Further variations include creating a puncture in the patient's scalp, and inserting a guide tube into the puncture in the patient's scalp. Another variation includes stabilizing the scalp using a stabilizer coupled to the guide tube. A further variation includes drilling, through the guide tube, a hole in the patient's skull. Another variation includes inserting a plug into the first receptacle in the base, such that a top surface of the plug is substantially flush with a top surface of the base. In another variation, the inserting includes press-fitting the plug into the first receptacle in the base. A further variation includes removing the plug from the first receptacle in the base by driving a tool in or adjacent to a soft portion of the plug. Yet another variation includes removing the anchoring base from the skull by inserting a tool into a second receptacle in the top portion of the base, wherein the second receptacle in the top portion of the base is different from the first receptacle in the base.

In a third example, this document discusses, among other things, a cap. The cap is sized and shaped to be located over an anchoring base of a fiducial marker. The cap includes an underside conforming to one or more features of the anchoring base. The cap includes a topside providing a downward taper toward the underside that is gradual enough to reduce or avoid discomfort to a portion of a patient's scalp near the fiducial marker base. Other aspects of the present systems, devices, and methods will become apparent upon reading the following detailed description and viewing the drawings that form a part thereof.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a cross-sectional schematic diagram illustrating generally, by way of example, but not by way of limitation, one embodiment of an anchoring base portion of a fiducial marker assembly, and portions of an environment in which it is used.

FIG. 2 is a schematic diagram illustrating generally, by way of example, but not by way of limitation, one embodiment of a top view of the base illustrated in FIG. 1.

FIG. 3 is a cross-sectional schematic diagram illustrating generally, by way of example, but not by way of limitation, one embodiment in which a fiducial marker assembly includes a imagable or otherwise locatable spherical or other fiducial marker at a proximal end of a downwardly extending shaft.

FIG. 4 is a cross-sectional schematic diagram illustrating generally, by way of example, but not by way of limitation, one embodiment in which a fiducial marker assembly includes a registration receptacle at a proximal end of a downwardly extending shaft.

FIG. 5 is a cross-sectional schematic diagram illustrating generally, by way of example, but not by way of limitation, one embodiment in which a fiducial marker assembly includes a plug sized and shaped and sufficiently compliant to be press-fit into base receptacle to prevent the accumulation of biological material or other debris therein when neither the fiducial marker nor the registration receptacle is screwed into the base receptacle.

FIG. 6 is a cross-sectional and side view schematic diagram illustrating generally, by way of example, but not by way of limitation, one embodiment of an alternative base having a substantially cylindrical externally-threaded outer portion and a blunt bottom portion.

FIG. 7 is a side view schematic diagram illustrating generally, by way of example, but not by way of limitation, one embodiment of a guide tube for assisting in disposing a base.

FIG. 8 is a side view schematic diagram illustrating generally, by way of example, but not by way of limitation, insertion of a sharp instrument such as a trocar or the like through a lumen of a guide tube for piercing a portal in a scalp.

FIG. 9 is a side view schematic diagram illustrating generally, by way of example, but not by way of limitation, one embodiment in which a beveled distal tip of a guide tube is inserted into a scalp portal.

FIG. 10 is a side view schematic diagram illustrating generally, by way of example, but not by way of limitation, one embodiment in which a drill bit is inserted through a lumen of a guide tube for drilling into a skull while a scalp is being stabilized by a slidable or a fixed flange.

FIG. 11 is a side view schematic diagram illustrating generally, by way of example, but not by way of limitation, one embodiment in which a base is inserted into the drilled-out portion of a skull such that a top portion of the base is flush with or recessed from an outer surface of the skull.

FIG. 12A is a cross-sectional view schematic diagram illustrating generally, by way of example, but not by way of limitation, one embodiment of a screw-in base including a flange located above the top surface of a skull and an externally-threaded shaft extending outwardly above the top surface of the skull for receiving a screw-on fiducial marker, a registration receptacle, or the like.

FIG. 12B is a cross-sectional view schematic diagram further illustrating generally, by way of example, but not by way of limitation, one embodiment of an atraumatic cap overlying a raised portion of a shaft.

FIG. 13 is a schematic diagram illustrating generally, by way of example, but not by way of limitation, an alternative embodiment of a base in which the base receptacle includes an alternative engagement mechanism; FIG. 13 also illustrates a compatible registration receptacle assembly, fiducial marker assembly, and base insertion tool portion

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the scope of the present invention. The following detailed description is, therefore, not to be taken in a limiting sense, and the scope of the present invention is defined by the appended claims and their equivalents.

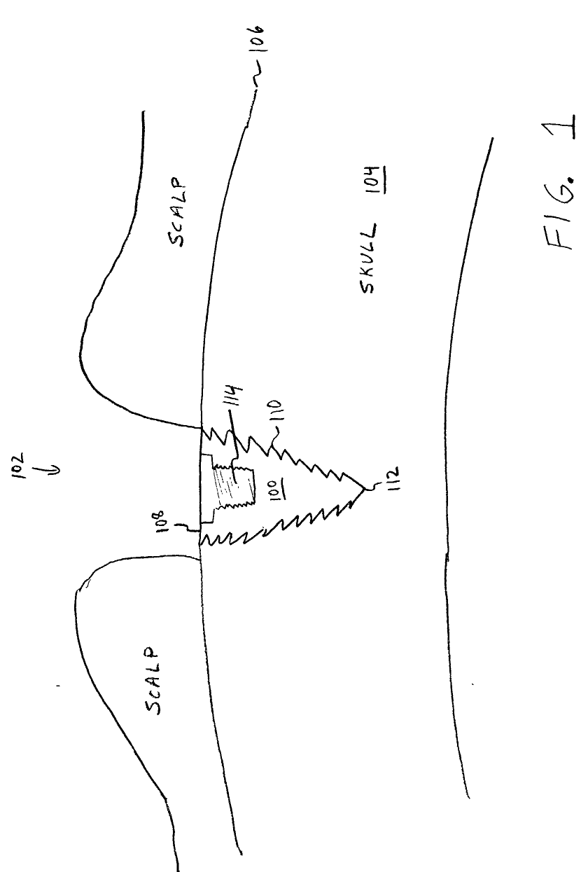

In this document, the terms “a” or “an” are used, as is common in patent documents, to include one or more than one. Furthermore, all publications, patents, and patent documents referred to in this document are incorporated by reference herein in their entirety, as though individually incorporated by reference. In the event of inconsistent usages between this documents and those documents so incorporated by reference, the usage in the incorporated reference(s) should be considered supplementary to that of this document; for irreconcilable inconsistencies, the usage in this document controls FIG. 1 is a cross-sectional schematic diagram illustrating generally, by way of example, but not by way of limitation, one embodiment of an anchoring base 100 portion of a fiducial marker assembly 102, and portions of an environment in which fiducial marker assembly 102 is used. In the example of FIG. 1, base 100 is sized and shaped for being implanted in a patient's skull 104, either flush with, or recessed from, an outer surface 106 of skull 104. For example, as illustrated in FIG. 1, base 100 does not include any lip or shaft extending upward from outer surface 106 of skull 104. Instead, top portion 108 of base 100 is sized and shaped and threaded such that it can be implanted either flush with, or recessed from, an outer surface 106 of skull 104. In this example, base 100 includes a self-tapping or other externally threaded outer portion 110 extending distally outward from top portion 108 of base 100. In one embodiment, outer portion 110 of base 100 is conically-tapered toward a relatively sharp distal tip 112, as illustrated in FIG. 1, thereby allowing self-drilling, such as by using a manual or power-driven insertion tool. In another embodiment, outer portion 110 of base 100 is substantially cylindrical, such that it terminates at a relatively flat distal tip 112. In such an embodiment, base 100 may, but need not, include self-tapping external threads. In the example illustrated in FIG. 1, base 100 also includes an internally threaded receptacle 114 extending distally into base 100 from top portion 108 of base 100.

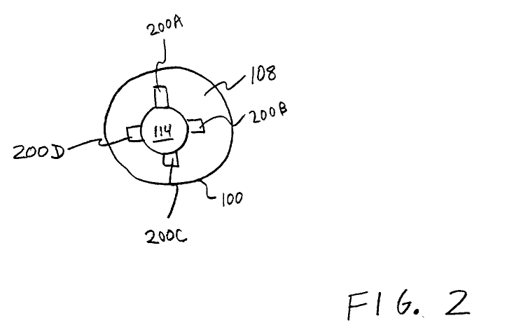

FIG. 2 is a schematic diagram illustrating generally, by way of example, but not by way of limitation, one embodiment of a top view of the base 100 illustrated in FIG. 1. FIG. 2 illustrates receptacle 114 in top surface 108 of base 100, together with at least one tool-receiving receptacle (such as a slot, hex receptacle, keyhole, or the like) for unscrewing base 100 from skull 104 (or, alternatively, for screwing base 100 into skull 104). In this example, the illustrated tool-receiving receptacle includes four slots 200A-D, such as for receiving portions of a Phillips-type screwdriver tip therein for unscrewing base 100 from skull 104. However, other examples could include two slots 200 or a different number of slots 200.

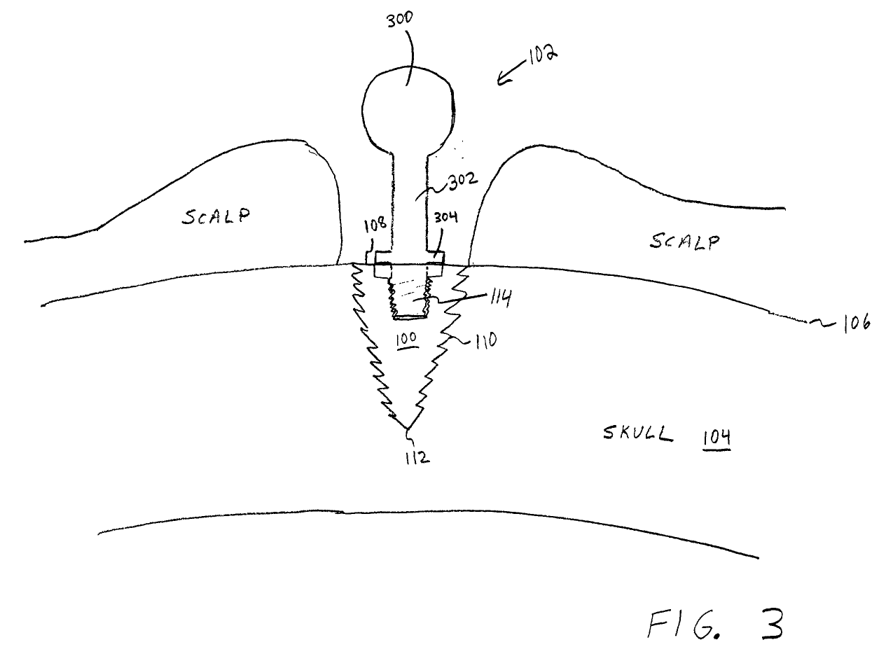

FIG. 3 is a cross-sectional schematic diagram illustrating generally, by way of example, but not by way of limitation, one embodiment in which fiducial marker assembly 102 includes a imagable or otherwise locatable spherical or other fiducial marker 300 at a proximal end of a downwardly extending shaft 302. Shaft 302 includes an externally threaded distal portion that is sized and shaped to be screwed into receptacle 114 of base 100. In one example, shaft 302 also includes a flange or other stop 304 that limits the travel of the distal end of shaft 302 into receptacle 114, thereby defining the height of the center of spherical fiducial marker 300 from stop 304. In another example, stop 304 is omitted, such that completely screwing shaft 302 into receptacle 114 defines a height of the center of spherical fiducial marker from the bottom of receptacle 114. Illustrative examples of marker 300, include a magnetic resonance imaging (MRI) visible marker for use in obtaining preoperative or other MRI images, a computed tomography (CT) visible marker for use in obtaining preoperative or other CT images, an X-ray visible marker for use in obtaining preoperative or other radiographic images, and a light or other electromagnetic radiation emitting (or reflective) marker for serving as a locatable fiducial marker during patient registration or subsequent surgical intervention in the operating room.

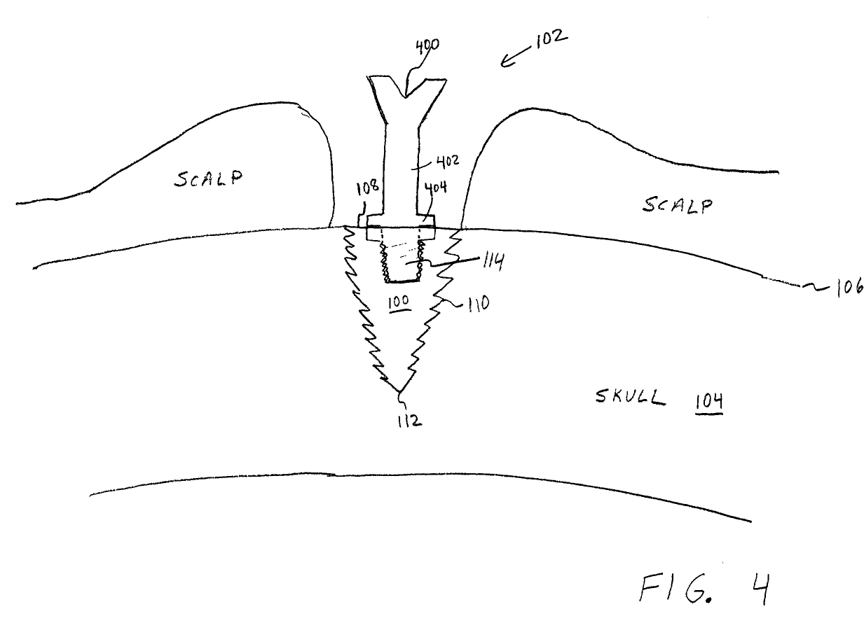

FIG. 4 is a cross-sectional schematic diagram illustrating generally, by way of example, but not by way of limitation, one embodiment in which fiducial marker assembly 102 includes a registration receptacle 400 at a proximal end of a downwardly extending shaft 402. Shaft 402 includes an externally threaded distal portion that is sized and shaped to be screwed into receptacle 114 of base 100. In one example, shaft 402 also includes a flange or other stop 404 that limits the travel of the distal end of shaft 402 into receptacle 114, thereby defining the height of the center (e.g., point of maximum depression) of registration receptacle 400 from stop 304 to be the same as the height of the center of fiducial marker 300 from stop 304. In another example, stop 404 is omitted, such that completely screwing shaft 402 into receptacle 114 defines a height of the center of the registration receptacle 400 from the bottom of base receptacle 114. In one example, registration receptacle 400 is sized and shaped to receive a sharp tip portion of a wand used in the operating room in conjunction with the image-guided workstation.

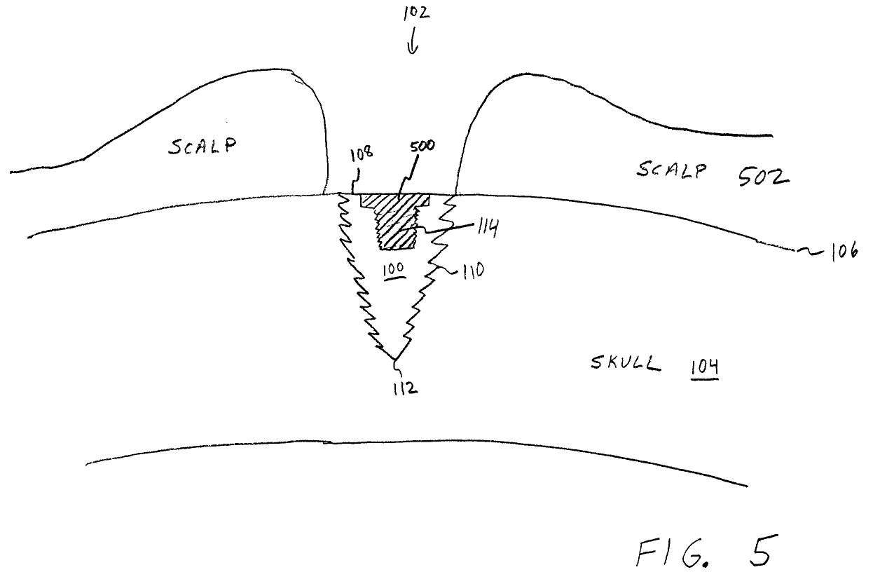

FIG. 5 is a cross-sectional schematic diagram illustrating generally, by way of example, but not by way of limitation, one embodiment in which fiducial marker assembly 102 includes a plug 500, sized and shaped and sufficiently compliant to be press-fit into base receptacle 114 to prevent the accumulation of biological material or other debris therein when neither fiducial marker 300 or registration receptacle 400 is screwed into receptacle 114. In this example, plug 500 is sized and shaped to be flush with top portion 108 of base 100. In one embodiment, plug 500 includes at least one soft portion that is sufficiently compliant to allow a needle or other tool to pierce or otherwise be inserted into and/or along plug 500 to pull plug 500 out of base receptacle 114. In one example, plug 500 also includes portions that are sized and shaped to fill slots 200A-D, as well as an interior portion of base receptacle 114. In one operative example, a physician press-fits plug 500 into place before suturing scalp 502 closed. This may be desirable, for example, between preoperative imaging and the subsequent surgical procedure, which may be separated by an arbitrarily long period of time.

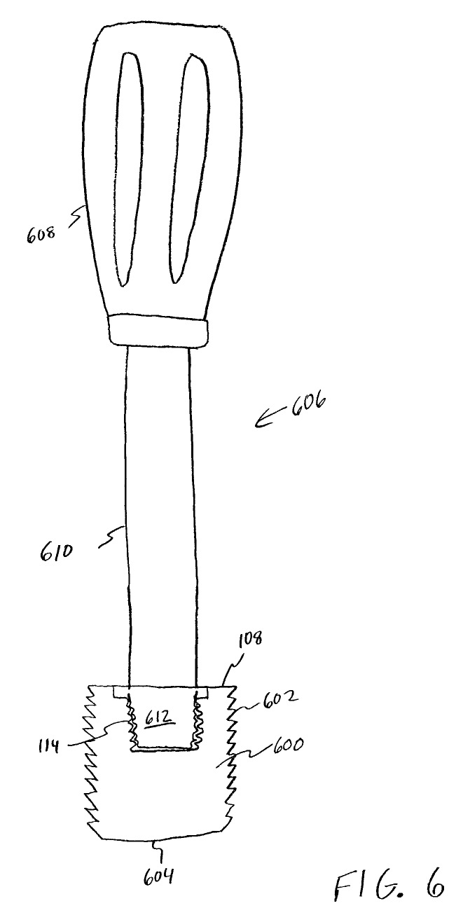

FIG. 6 is a cross-sectional and side view schematic diagram illustrating generally, by way of example, but not by way of limitation, one embodiment of an alternative base 600 having a substantially cylindrical externally-threaded outer portion 602 and blunt bottom portion 604, as discussed above. In this example, base 600 also includes base receptacle 114 and top portion 108. FIG. 6 also illustrates a base insertion tool 606, which includes a proximal handle 608, a shaft 610, and an externally-threaded distal tip 612 sized and shaped to be threadedly received into base receptacle 114. In one example handle 608 is detachable from shaft 610. In one example, base insertion tool 606 is threaded (e.g., clockwise) into base receptacle 114. Base insertion tool 606 is then used to thread base 600 (e.g., clockwise) into a portion drilled in skull 104 such that top portion 108 of base 600 is flush to or recessed from a top surface 106 of skull 104. Base insertion tool 606 is then unthreaded (e.g., counter-clockwise) from base receptacle 114.

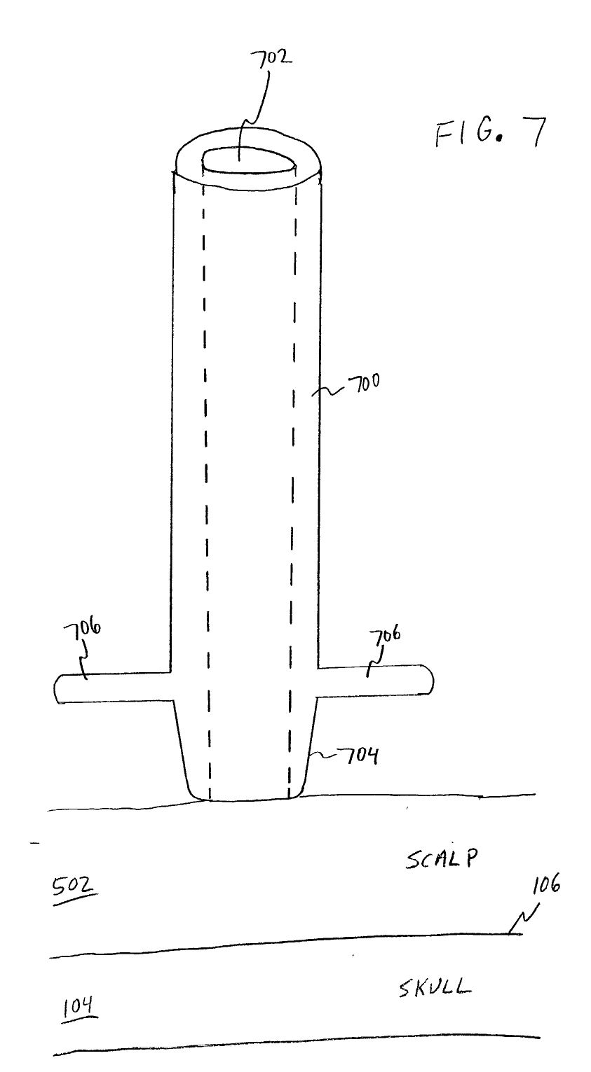

FIG. 7 is a side view schematic diagram illustrating generally, by way of example, but not by way of limitation, one embodiment of a guide tube 700 for assisting in disposing base 100 and/or base 600. Guide tube 700 includes a lumen 702 extending longitudinally therethrough. Lumen 702 is sized and shaped to allow base 100 and/or base 600 to pass therethrough. In this example, guide tube 700 includes a beveled distal tip 704, which is sized and shaped to align lumen 702 of guide tube 700 to a portal in the patient's scalp. In this illustrative example, guide tube 700 also includes a flange 706 extending radially outward circumferentially around a portion of guide tube 700 near beveled distal tip 704. In one example, flange 706 is fixedly positioned at a distance from beveled distal tip 704; this distance is selected such that flange 706 stabilizes a portion of the patient's scalp near the portal therein when beveled distal tip 704 of guide tube 700 is pressed into a portion of the portal in the patient's scalp. In another example, flange 706 is slidable longitudinally along guide tube 700 (e.g., like a washer, or the like, circumferentially surrounding guide tube 700) such that, by pushing downward on slidable flange 706, the physician can stabilize the portion of the patient's scalp near the portal therein when beveled distal tip 704 of guide tube 700 is pressed into a portion of the portal in the patient's scalp.

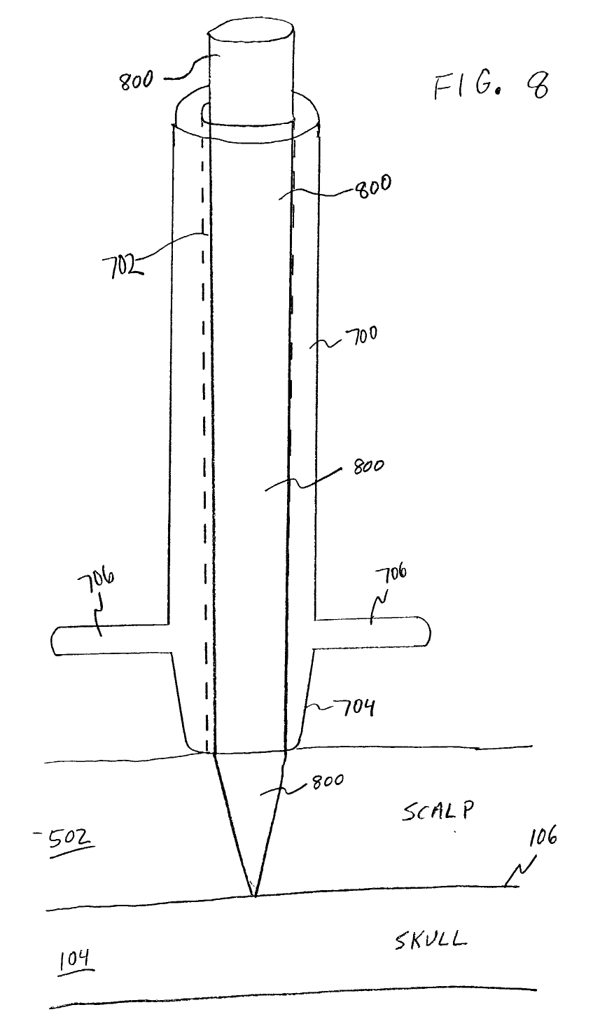

FIG. 8 is a side view schematic diagram illustrating generally, by way of example, but not by way of limitation, insertion of a sharp instrument such as a trocar 800 or the like through lumen 702 of guide tube 700 for piercing a portal in scalp 502.

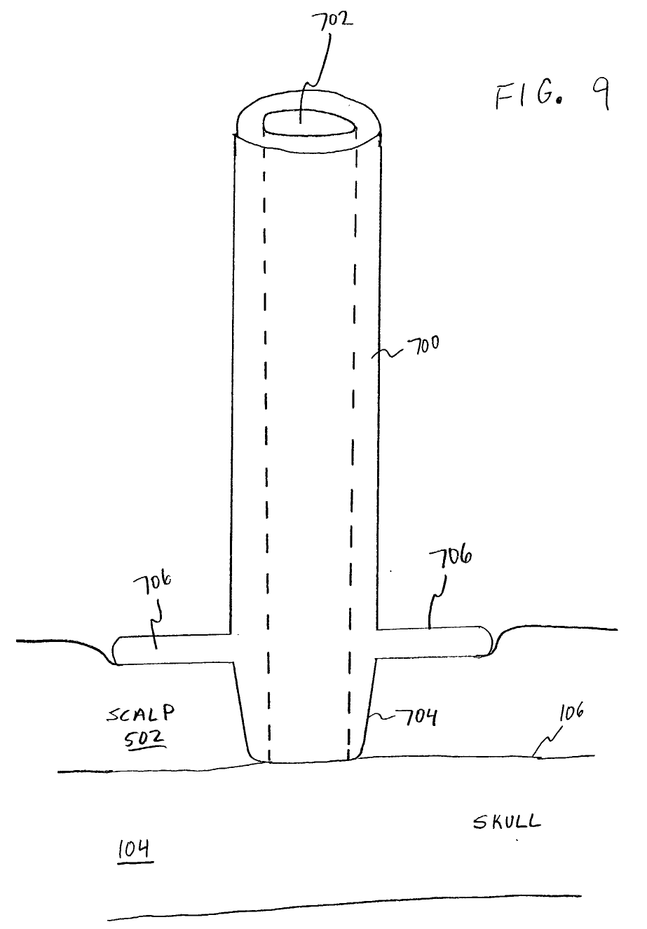

FIG. 9 is a side view schematic diagram illustrating generally, by way of example, but not by way of limitation, one embodiment in which beveled distal tip 704 of guide tube 700 inserted into the portal in scalp 502 that was created by trocar 800. Flange 706 stabilizes a portion of scalp 502 around the portal, either by virtue of its distance from the beveled distal tip 704, or by virtue of flange 706 being slidably pushed downward by the physician.

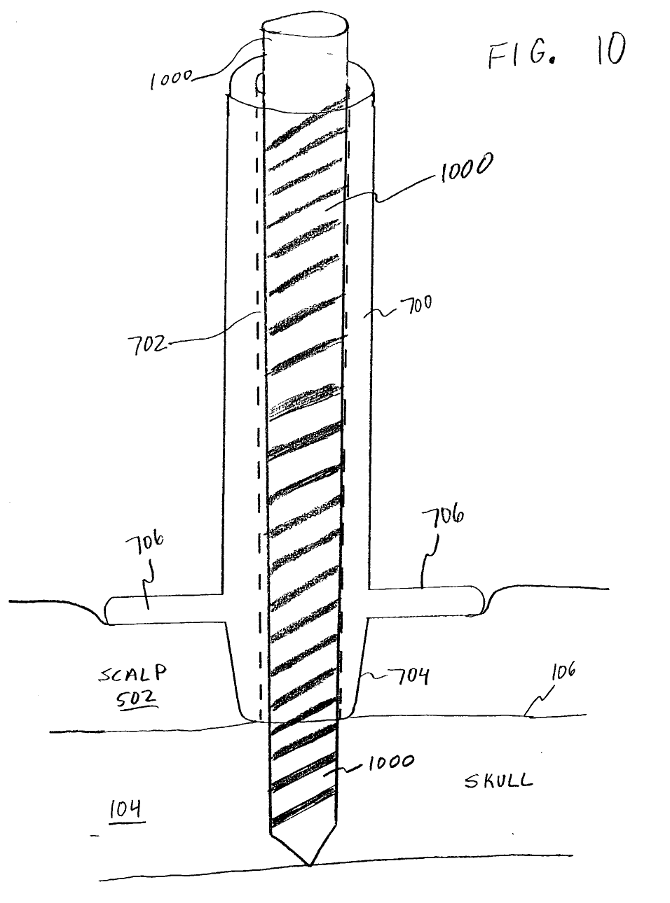

FIG. 10 is a side view schematic diagram illustrating generally, by way of example, but not by way of limitation, one embodiment in which drill bit 1000 is inserted through lumen 702 of guide tube 700 for drilling into skull 104 while a portion of scalp 502 is being stabilized by slidable or fixed flange 706.

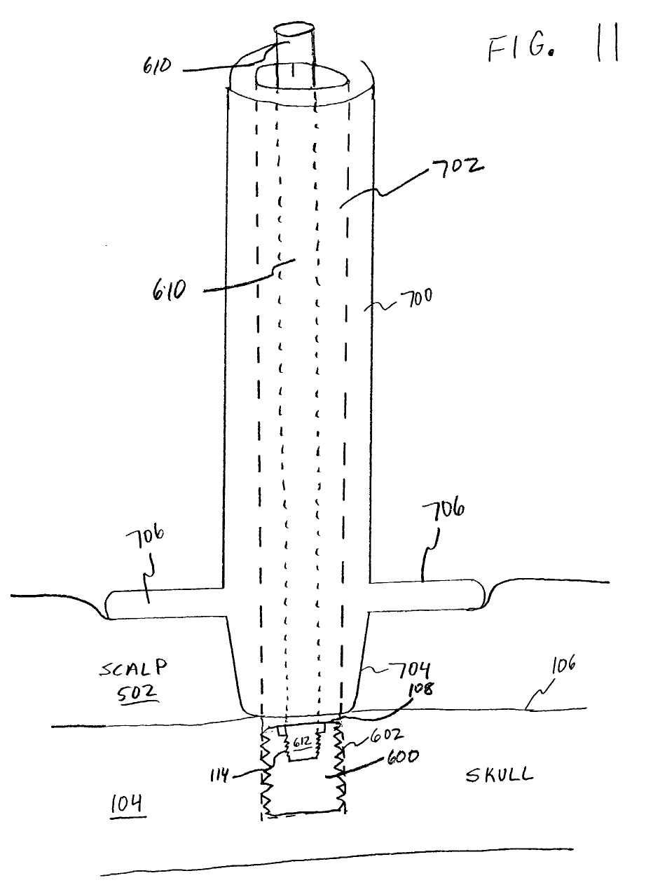

FIG. 11 is a side view schematic diagram illustrating generally, by way of example, but not by way of limitation, one embodiment in which base 600 is inserted into the drilled-out portion of skull 104 such that top portion 108 is flush with or recessed from outer surface 106 of skull 104. In this example, base 600 is first screwed onto distal tip 612 of base insertion instrument shaft 610, then inserted through lumen 702 of guide tube 700. Base 600 is then threaded into the drilled-out portion of skull 104 by screwing it in using base insertion instrument 606, while scalp 502 is being stabilized by flange 706. Distal tip 612 of shaft 610 of base insertion instrument 606 is then unscrewed from base 600, and shaft 610 is withdrawn from lumen 702 of guide tube 700.

Although FIGS. 10 and 11 illustrate drilling out a portion of skull 104 to insert a base 600, alternatively, a self-drilling base (e.g., base 100) is used, so that no separate drilling step is required. Self-drilling base 100 is placed on the distal tip 612 of base insertion instrument 606, which may include a power-driven screwdriver to rotate shaft 610 of base insertion instrument 606, so as to screw base 100 into skull 104, such that top portion 108 of base 100 is flush with or recessed from outer surface 106 of skull 104. This flush or recessed mounting improves patient comfort, particularly if scalp 502 is to be sewn up, such as where there is an extended period of time between preoperative imaging and the subsequent surgical procedure.

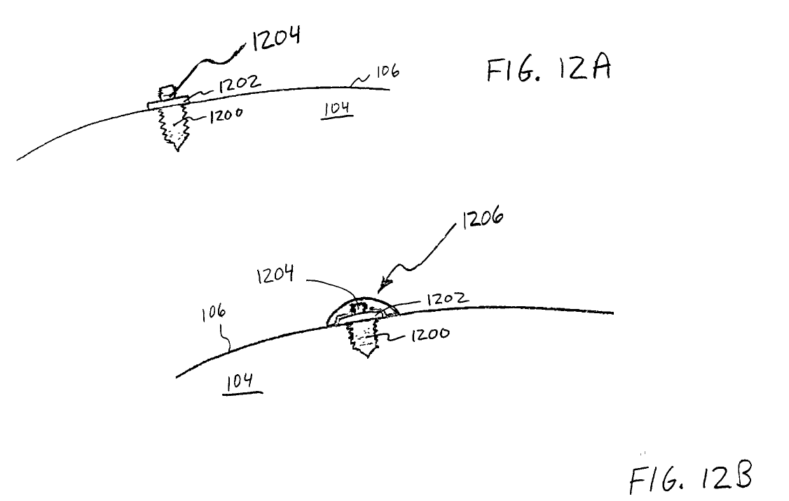

FIGS. 12A and 12B illustrate an alternative solution to providing patient comfort. FIG. 12A is a cross-sectional view schematic diagram illustrating generally, by way of example, but not by way of limitation, one embodiment of a screw-in base 1200 including a flange 1202 located above the top surface 106 of skull 104 and an externally-threaded shaft 1204 extending outwardly above top surface 106 of skull 104 for receiving a screw-on fiducial marker, a registration receptacle, or the like. FIG. 12B is a cross-sectional view schematic diagram further illustrating generally, by way of example, but not by way of limitation, one embodiment of an atraumatic cap 1206 overlying the raised portion of shaft 1204. In one example, cap 1206 is made of a material that is compliant enough to allow it to be press-fit onto and over shaft 1204 and flange 1202. In this example, the underside of cap 1206 is sized and shaped to be conformal to shaft 1204, flange 1202, and any other features of the fiducial marker base being covered. The top of cap 1206 is hemispherically or otherwise tapered at an acute enough angle with top surface 106 of skull 104 such that discomfort to nearby portions of the patient's scalp 502 is reduced or avoided altogether.

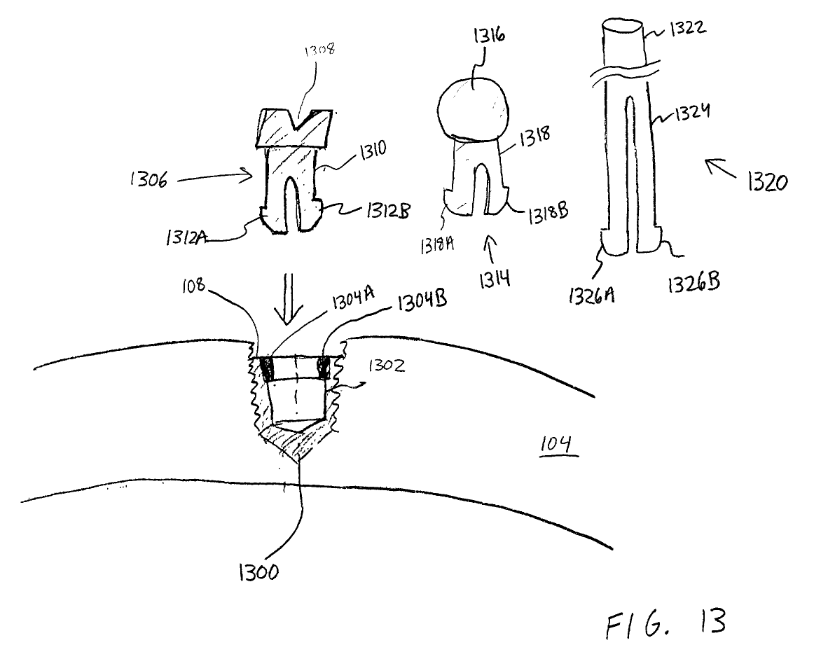

FIG. 13 is a schematic diagram illustrating generally, by way of example, but not by way of limitation, an alternative embodiment of base 1300 in which base receptacle 1302 is not internally threaded, but instead includes an alternative engagement mechanism. In this example, the alternative engagement mechanism includes female snap-fit receptacles 1304A-B formed into the sidewalls of the interior of base receptacle 1302. FIG. 13 also illustrates a registration receptacle assembly 1306, including a divot-like registration receptacle 1308, a shaft 1310, and male snap-fit protrusions 1312A-B configured to be snap-fit into corresponding female snap-fit receptacles 1304A-B of base receptacle 1302. (Of course, male and female snap-fit connections can be interchanged such that base receptacle 1302 includes male snap-fit protrusions configured for receiving female snap-fit receptacles thereupon.) A portion of shaft 1310 is split, providing sufficient compliance to permit the snap-fit operation. FIG. 13 also illustrates a fiducial marker assembly 1314 including a locatable fiducial marker 1316, a split shaft 1318, and male protrusions or other snap-fit features 1318A-B for engaging corresponding mating features (e.g., 1304A-B) in base receptacle 1302. FIG. 13 also illustrates a portion of a base insertion tool 1320 (analogous to 606), including a shaft 1322 having a split-shaft portion 1324, and male protrusions or other snap-fit features 1318A-B for engaging corresponding mating features (e.g., 1304A-B) in base receptacle 1302. Base receptacle 1302 need not be limited to threaded and snap-fit engagement devices, but could include any other known engagement devices or structures.

In a further example, a trajectory guide can be mounted to one or more of the bases described herein, such as by using a suitably sized and shaped screw or press-fit bolt that couples a base portion of the trajectory guide to the base receptacle. One embodiment of a suitable ball-and-socket trajectory guide is described in Truwit U.S. Pat. No. 6,267,769, the disclosure of which is incorporated by reference herein in its entirety, including its discussion of a ball-and-socket trajectory guide. Another example of a suitable trajectory guide is described in Skakoon et al. U.S. patent application Ser. No. 09/828,451 (Attorney Docket No. 00723.031 US1), filed on Apr. 6, 2001 and assigned to Image-Guided Neurologics, Inc., the disclosure of which is incorporated herein by reference in its entirety, including its disclosure of a rotatable saddle trajectory guide.

It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects.

What is claimed is:

1. A system comprising:
an anchoring base, sized and shaped to be implanted within in a patient's skull such that a top portion of the base is flush to or recessed from an outer surface of the patient's skull, the base including:
an externally threaded outer portion extending from the top portion of the base; and
an engagable base receptacle extending from the top portion of the base;
a locatable fiducial marker, including a shaft portion sized and shaped to be received and engaged into the base receptacle; and
a registration receptacle, including a shaft portion sized and shaped to be received and engaged into the base receptacle.

2. The system of claim 1, in which the top portion of the base includes at least one tool-receiving receptacle in addition to the base receptacle.

3. The system of claim 2, in which the top portion of the base includes at least one slot.

4. The system of claim 3, in which the top portion of the base includes a plurality of screwdriver slots.

5. The system of claim 1, in which the locatable fiducial marker is imagable by at least one of: magnetic resonance imaging (MRI), computed tomography (CT), X-ray radiography, a light detector, and an electromagnetic field detector.

6. The system of claim 1, further comprising a plug sized and shaped to be received in base receptacle.

7. The system of claim 6, in which the plug is constructed to allow the plug to be press-fit into the base receptacle.

8. The system of claim 7, in which the plug is sufficiently compliant to allow the plug to be removed from the base receptacle by inserting a tool into the plug to assist in pulling the plug out of the base receptacle.

9. The system of claim 6, in which the plug is sized and shaped to provide a top surface that is substantially flush with the top surface of the base receptacle.

10. The system of claim 1, further including a base insertion tool, the base insertion tool comprising:
an engaging portion sized and shaped to be received and engaged within the base receptacle; and
a shaft, coupled to and extending outwardly from the engaging portion.

11. The system of claim 10, further including a guide tube, including a lumen extending longitudinally therethrough, the lumen sized and shaped to allow the base to pass through the lumen, and the guide tube including a beveled distal tip, sized and shaped to align the guide tube lumen to a portal in the patient's scalp.

12. The system of claim 11, in which the guide tube further includes a flange extending outwardly from the guide tube near the beveled distal tip, at a distance from the beveled distal tip that is selected such that the flange stabilizes a portion of the patient's scalp near the portal in the patient's scalp when the beveled distal tip of the guide tube is pressed into a portion of the portal in the patient's scalp.

13. The system of claim 12, in which the flange is longitudinally slidable along the guide tube to select the distance from the beveled distal tip to stabilize a portion of the patient's scalp.

14. The system of claim 11, further including a trocar sized and shaped to be received within the lumen of the guide tube.

15. The system of claim 11, further including a drill bit sized and shaped to be received within the lumen of the guide tube, the drill bit having an outer diameter that is sized and shaped to create a portal in the patient's skull, wherein the portal is sized to receive the externally threaded portion of the base in a self-tapping manner.

16. The system of claim 1, in which the base receptacle is internally threaded to provide an engagement mechanism for the base.

17. The system of claim 1, in which the base receptacle includes at least one of a male snap-fit coupling and a female snap-fit coupling to provide an engagement mechanism for the base.

18. The system of claim 1, further including a trajectory guide coupled to the engagable base receptacle.

19. A method comprising:
threading an anchoring base into a patient's skull such that a top portion of the base is flush with or recessed from an outer portion of the patient's skull;
coupling a locatable fiducial marker to a first receptacle in the base;
obtaining an image the patient's skull such that the locatable fiducial marker is apparent on the image;
removing the locatable fiducial marker from first receptacle in the base;
coupling a registration receptacle to the first receptacle in the base such that a portion of the registration receptacle is in a predetermined spatial relationship to a portion of the locatable fiducial marker when the locatable fiducial marker was coupled to the base; and
registering a location of the portion of the registration receptacle to the portion of the locatable fiducial marker that is apparent on the image.

20. The method of claim 19, in which the threading the anchoring base into a patient's skull comprises:
engaging an internal portion of the first receptacle in the base onto a tool;
screwing external threads of the base into the patient's skull; and
disengaging the tool from the first receptacle in the base.

21. The method of claim 20, in which the engaging the internal portion of the first receptacle in the base onto a tool includes threading internal threads of the first receptacle in the base onto external threads on the tool.

22. The method of claim 20, in which the engaging the internal portion of the first receptacle in the base onto a tool includes snap fitting an internal portion of the first receptacle in the base onto an external portion of the tool.

23. The method of claim 19, further comprising:
creating a puncture in the patient's scalp; and
inserting a guide tube into the puncture in the patient's scalp.

24. The method of claim 23, further comprising stabilizing the scalp using a stabilizer coupled to the guide tube.

25. The method of claim 23, further comprising drilling, through the guide tube, a hole in the patient's skull.

26. The method of claim 19, further including inserting a plug into the first receptacle in the base, such that a top surface of the plug is substantially flush with a top surface of the base.

27. The method of claim 26, in which the inserting includes press-fitting the plug into the first receptacle in the base.

28. The method of claim 26, further including removing the plug from the first receptacle in the base by driving a tool in or adjacent to a soft portion of the plug.

29. The method of claim 19, further including removing the anchoring base from the skull by inserting a tool into a second receptacle in the top portion of the base, wherein the second receptacle in the top portion of the base is different from the first receptacle in the base.

30. The method of claim 19, further including coupling a trajectory guide to the anchoring base.

31. A cap, sized and shaped to be located over an anchoring base of a fiducial marker, the cap including an underside conforming to one or more features of the anchoring base, the cap including a topside providing a downward taper toward the underside that is gradual enough to reduce or avoid discomfort to a portion of a patient's scalp near the fiducial marker base.
